(19) United States
(12) Patent Application Publication
(10) Pub. No.: US 2023/0172230 A1
(43) Pub. Date: Jun. 8, 2023

(54) ENZYME COMPOSITIONS AND RELATED METHODS

(71) Applicant: Premex, Inc., Durham, NC (US)

(72) Inventors: Alejandro Mesa GOMEZ, Durham, NC (US); Milena Agudelo PALACIO, Durham, NC (US)

(21) Appl. No.: 18/074,931

(22) Filed: Dec. 5, 2022

Related U.S. Application Data
(60) Provisional application No. 63/285,538, filed on Dec. 3, 2021.

(51) Int. Cl. A23K 10/14; C12N 9/16

(52) U.S. Cl. CPC A23K 10/14; C12Y 301/03; C12N 9/16; A23K 50/30

(57) ABSTRACT

This disclosure is directed to methods of formulating a customized animal feed that includes a customized enzyme composition. Methods of achieving one or more zootechnical objectives for an animal are also provided.

### CROSS-REFERENCE TO RELATED APPLICATIONS

The present application claims priority to U.S. provisional application No. 63/285,538 filed Dec. 3, 2021, the contents of which are incorporated herein.

### BACKGROUND OF THE DISCLOSURE

Exogenous enzymes have historically been considered as one of the most frequent tools in the search for improvements in nutrient digestibility and control of anti-nutritional factors in diets. Although their use has been extended in the animal nutrition market, the use of exogenous enzymes is still limited by several market components, including the fact that the technical information derived from the enzyme functionality is controlled by the suppliers. The enzyme suppliers may limit technical and share only those data that represent a competitive advantage, while restricting the dose, the nutritional matrix and the real stability conditions of their products. As a result, there is a need to create solutions oriented to objectify the comparison of enzymes by analyzing the enzymes under the same parameters.

### SUMMARY OF THE DISCLOSURE

According to one aspect, a method of formulating a customized animal feed is provided. The method includes the steps of:

receiving animal owner data;

developing one or more zootechnical objectives to be obtained based on administration of a customized animal feed, the customized animal feed comprising one or more feed components and a customized enzyme composition;

characterizing feed raw materials, the raw materials including one or more feed components and one or more enzymes;

generating, by processor, an enzyme selection index;

assigning, by processor, an enzyme performance score;

predicting, by processor, the customized enzyme composition comprising one or more enzymes for suitability in achieving the one or more zootechnical objectives;

predicting, by processor, a recipe for the customized animal feed;

manufacturing the customized enzyme composition;

manufacturing the customized animal feed;

administering the customized animal feed to an animal;

evaluating the animal health and performance after administration of the customized animal feed.

According to one embodiment, the animal owner data includes animal nutritional needs, animal weight, animal activity level, ammonia production level of the animal, animal body temperature, ambient temperature of a facility where the animal is kept, humidity, animal weight, animal water intake, animal body pH, or a combination thereof. According to one embodiment, the one or more zootechnical objectives includes improving animal health and performance. According to one embodiment, the method of formulating a customized animal feed further includes the step of quantifying feed components, evaluating enzyme characteristics of enzymes that may be utilized in enzyme compositions, and assessing diet formulation for maximum efficiency. According to one embodiment, the method of formulating a customized animal feed further includes the step of transmitting the customized animal feed recipe to the animal owner for acceptance or denial by the animal owner, wherein upon approval by the animal owner, the customized animal feed recipe is transmitted to an animal feed manufacturer.

According to one aspect, a method of achieving one or more zootechnical objectives is provided. The method includes the steps of:

developing one or more zootechnical objectives to be obtained based on administration of an animal feed comprising one or more feed components and a customized enzyme composition;

analyzing and selecting, by processor, one or more feed components and enzymes for suitability in achieving the one or more zootechnical objectives, including nutritional intake;

predicting, by processor, the customized enzyme composition comprising one or more enzymes for suitability in achieving the one or more zootechnical objectives;

predicting, by processor, a recipe for an animal feed comprising one or more feed components and the customized enzyme composition;

manufacturing the customized enzyme composition;

manufacturing the customized animal feed;

feeding or administering the customized animal feed to the at least one animal;

evaluating impact on the one or more zootechnical objectives based on feeding the customized animal feed to the at least one animal; and

optionally, implementing one or more design changes to the customized enzyme composition, feed components, or a combination thereof based on the step of evaluating impact on the one or more zootechnical objectives. According to one embodiment, the one or more zootechnical objectives includes improving animal health and performance. According to one embodiment, the customized animal feed and customized enzyme composition are combined prior to feeding to the at least one animal. According to one embodiment, the step of evaluating impact on the one or more zootechnical objectives includes the step of analyzing, by processor, animal weight, animal activity level, ammonia production level of the animal, animal body temperature, environmental temperature, humidity, animal weight, animal water intake, animal body pH, or a combination thereof. According to one embodiment, the method of achieving one or more zootechnical objectives further includes the step of transmitting the customized animal feed recipe to the animal owner for acceptance or denial by the animal owner, wherein upon approval by the animal owner, the customized animal feed recipe is transmitted to an animal feed manufacturer.

According to one aspect, a method of manufacturing an enzyme composition for achieving one or more zootechnical objectives is provided. The method includes the steps of:

developing one or more zootechnical objectives to be obtained based on administration of an animal feed comprising one or more feed components and a customized enzyme composition;

comparing enzyme characteristics of commercially available enzymes for inclusion in customized enzyme composition;

predicting, by processor, the customized enzyme composition comprising one or more enzymes for suitability in achieving the one or more zootechnical objectives;

predicting, by processor, a recipe for an animal feed comprising one or more feed components and the customized enzyme composition; and

manufacturing the customized enzyme composition for achieving one or more zootechnical objectives, upon administration. According to one embodiment, the one or more zootechnical objectives includes improving animal health and performance.

According to one aspect, an animal feed additive is provided that includes an enzyme composition as provided herein.

According to one aspect, an enzyme composition for feeding to an animal to achieve one or more zootechnical objectives, the customized enzyme composition includes at least one enzyme; and optionally, one or more suitable carriers, wherein the at least one enzyme is selected to achieve the one or more zootechnical objectives.

According to one aspect, a system for achieving one or more zootechnical objectives is provided including:

at least one server;

at least one database;

at least one software package;

at least one animal sensor coupled to a gateway that is in wireless communication with the at least one server, database and software package;

a memory and processor in wireless communication with the server, database, software package and sensor, the memory and processor configured to perform the steps as provided herein. According to one embodiment, the software package is adapted to run at least one algorithm to assist in:

analyzing and selecting, by processor, one or more feed components and enzymes for suitability in achieving the one or more zootechnical objectives, including nutritional intake;

predicting, by processor, the customized enzyme composition comprising one or more enzymes for suitability in achieving the one or more zootechnical objectives;

predicting, by processor, a recipe for an animal feed comprising one or more feed components and the customized enzyme composition;

evaluating impact on the one or more zootechnical objectives based on feeding the customized animal feed to the at least one animal; or

a combination thereof. According to one embodiment, the system includes an animal owner's interface including a data entry system.

### BRIEF DESCRIPTION OF THE DRAWINGS

FIG. 1 provides one embodiment of a method of formulating a customized animal feed.

FIG. 2 provides one embodiment of a method of achieving one or more zootechnical objectives.

FIG. 3 provides a description on each treatment diet utilized in Experimental Example 1.

FIG. 4 provides the results of Experimental Example 1 on various physical indicators in pigs.

FIG. 5 illustrates alpha diversity in feces by location for Experimental Example 2.

FIG. 6 illustrates the intestinal microbiome in terms of beta diversity for Experimental Example 2.

FIG. 7 illustrates in the histopathology scores (aggregated) in the cecum for Experimental Example 2.

FIG. 8 illustrates in the histopathology scores (aggregated) in the ileum for Experimental Example 2.

### DETAILED DESCRIPTION OF THE DISCLOSURE

The present disclosure will now be described more fully hereinafter with reference to exemplary embodiments thereof. These exemplary embodiments are described so that this disclosure will be thorough and complete, and will fully convey the scope of the disclosure to those skilled in the art. Indeed, the present disclosure may be embodied in many different forms and should not be construed as limited to the embodiments set forth herein; rather, these embodiments are provided so that this disclosure will satisfy applicable legal requirements.

The embodiments described below may assume various alternative orientations and step sequences, except where expressly specified to the contrary. Specific devices and any related processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

While the following terms are believed to be well understood by one of ordinary skill in the art, the following definitions are set forth to facilitate explanation of the presently disclosed subject matter.

As used in the specification, and in the appended claims, the singular forms “a”, “an”, “the”, include plural referents unless the context clearly dictates otherwise.

As used in the specification, and in the appended claims, the term “custom” and “customized” refers to an animal feed and enzyme composition that are modified to suit a particular task or objective for one or more animals.

As used in the specification, and in the appended claims, the term “zootechnical” refers to technology or data associated with animal handling, breeding, keeping and overall animal health or performance.

As used in the specification, and in the appended claims, the term “animal” refers to any animal in need to exogenous enzyme supplementation including, but not limited to, livestock and domesticated animals.

As used in the specification, and in the appended claims, the term “server” refers to a computer in a network utilized to process and provide data to other computers or components.

As used in the specification, and in the appended claims, the term “database” refers to a data structure or organized collection of electronic data that is electronically accessible.

As used in the specification, and in the appended claims, the phrase “improving livestock health and performance” refers to the ability of the customized enzyme compositions, feed and methods provided herein to positively impact at least one metric, indicator or task associated with health, performance, or both health and performance.

### Overview of Systems and Methods

Enzymes depend on and interact with specific substrates. The variability of the raw materials included in an enzyme composition (and corresponding animal feed) as well as the possible metabolic interactions that may occur as a result of these variabilities may be analyzed or otherwise assessed to generate a customized enzyme composition that adapts to and addresses various nutritional needs of an animal. The customized enzyme composition may, thus, be customized or otherwise personalized based on the nutritional needs as well as per an animal owner's request or feedback. The nutritional needs may be addressed with or without restriction of species, physiological stage or production conditions. As a result, the enigmas that surround enzyme compositions added to animal feed may be unveiled and provide peace of mind and direction for an animal owner when making a decision regarding enzyme needs and consumption.

The present disclosure provides enzyme compositions and methods of evaluating feed production needs as well as developing and producing an enzyme composition to address such needs feed production needs. The methods provided herein produce and otherwise utilize a customized enzyme composition that is prepared based on a variety of factors including, but not limited to, a tailored characterization and nutrient opportunity analysis, the need for commercial products and other raw materials that can benefit the animals, the animal's environment, and an animal owner's input and needs. The customized enzyme compositions may be specific to an animal species at a physiological stage. Alternatively, the customized enzyme compositions may not be specific to an animal species and may be fed to a variety of animals. The customized enzyme compositions may be incorporated in an animal feed. The customized animal feed may be evaluated after administration to an animal with the goal of improving the customized animal feed and, in turn, animal health and performance.

The methods provided herein may utilize various system components (e.g., software, serves, processors, databases) to predict, specify and select enzymes suitable for an animal's health and performance needs. The methods may rely on various data including, but not limited to, data related to the functionality of enzymes, selection indexes to contrast commercial products, productive conditions in which use should be modulated, the maximum expected enzyme functionality, and the possible negative interactions that may occur through the administration of an enzyme composition. None of the prediction steps disclosed here may be performed by a human brain.

### System Components and Data Processing

A system for the various methods described herein is provided. According to one embodiment, a system is provided to aid in achieving one or more zootechnical objectives. According to one embodiment, a system is provided to formulate a customized animal feed.

According to one embodiment, the system includes at least one database. According to one embodiment, the at least one database integrates with software described herein. The at least one database may be coupled to or in wireless communication with a gateway that is, in turn, in wireless communication with the at least one server, processor and memory.

According to one embodiment, the system includes at least one server. The at least one server may be located in a cloud configuration. According to one embodiment, the system includes at least one computer coupled to the at least one server. The at least one computer may be located in a cloud configuration and coupled or in communication with the at least one server. The computer includes at least one processor and memory for analyzing the data as providing here and detecting at least one anomaly. According to one embodiment, one or more of the method steps described herein utilizes or otherwise relies on use of one or more of the system components.

A system for achieving one or more zootechnical objectives is provided. According to one embodiment, the system for achieving one or more zootechnical objectives includes at least one server; at least one database; at least one software package; at least one animal sensor coupled to a gateway that is in wireless communication with the at least one server, database and software package; a memory and processor in wireless communication with the server, database, software package and sensor. The memory, server and processor are configured to perform the method steps. According to one embodiment, the software package is adapted to run at least one algorithm to assist in: analyzing and selecting, by processor, one or more feed components and enzymes for suitability in achieving the one or more zootechnical objectives (including analyzing and selecting feed components and enzymes based on nutritional intake); predicting, by processor, the customized enzyme composition for suitability in achieving the one or more zootechnical objectives; predicting, by processor, a recipe for an animal feed comprising one or more feed components and the customized enzyme composition; evaluating impact on the one or more zootechnical objectives based on feeding the customized animal feed to the at least one animal; or a combination thereof. The prediction steps cannot be performed by a human brain. According to one embodiment, the system for achieving one or more zootechnical objectives includes an animal owner's interface including a data entry system.

A system for formulating a customized animal feed is provided. According to one embodiment, the system for formulating a customized animal feed includes at least one server; at least one database; at least one software package; at least one animal sensor coupled to a gateway that is in wireless communication with the at least one server, database and software package; a memory and processor in wireless communication with the server, database, software package and sensor. The memory, server and processor are configured to perform the method steps. According to one embodiment, the software package is adapted to run at least one algorithm to assist in: analyzing and selecting, by processor, one or more feed components and enzymes for suitability in formulating a customized animal feed (including analyzing and selecting feed components and enzymes based on nutritional intake); predicting, by processor, the customized enzyme composition; predicting, by processor, a recipe for an animal feed comprising one or more feed components and the customized enzyme composition; feeding the customized animal feed to the at least one animal; evaluating the customized animal feed; or a combination thereof. According to one embodiment, the system for formulating a customized animal feed includes an animal owner's interface including a data entry system.

According to one embodiment, a database is provided that stores and maintains various standardized data. Such standardized data may relate to health profiles of particular animal types and the genetic information of particular animal types. According to one embodiment, the database maintains one or more licenses to access one or more data services which aid in and provide data related to disease diagnosis, prescriptions medicines, health assessment data and genetic data. According to one embodiment, the aforementioned information is received, standardized or normalized and stored for later use. According to one embodiment, all standard data is cleaned and aggregated before further processing.

According to one embodiment, the software provided herein may be connected to or otherwise in communication with one or more databases where data of interest is stored, such as diet formulations, optimization programs at minimum cost or maximum efficiency, financial systems with details of product prices, reports of zootechnical indicators, monitoring systems of environmental conditions, and laboratory results for characterization of raw materials.

According to one embodiment, the at least one database stores and maintains data related to raw material characterization. Raw material data may include raw feed material nutrient substrates and substrate concentration data. Raw material data may contribute to a matrix of all feasible raw materials to be used in an animal feed and animal diet, indicating not only the nutrients required by nutritionists for optimization but also the enzyme substrates feasible to hydrolysis.

According to one embodiment, the at least one database stores and maintains data related to enzyme characterization and nutritional contribution. Such data may relate to data made available by commercial enzyme manufacturers including internal codes, prices, active ingredient concentration and nutritional matrix

According to one embodiment, the at least one database stores and maintains data related to supplier and animal owner enzyme use. Such supplier and customer enzyme data may include the identity of enzymes used by feed suppliers as well as animal owners. Consideration of such supplier and animal owner enzyme data ensures traceability and contributes to the prediction steps of the methods described herein.

According to one embodiment, the at least one database stores and maintains data related to enzyme-substrate interaction and relationships. According to one embodiment, the at least one database stores and maintains data related to species-enzyme relationship (including physiological phase and use efficiency data). According to one embodiment, the at least one database stores and maintains data related to definitions of antagonism and synergy relationships between raw materials, enzymes and substrates. The functionality of enzymes can be affected by external conditions that either generate a detriment in enzyme operation or potentiate enzyme effects. Data related to enzyme-substrate interaction and relationships may be utilized to calculate an expected functionality and modify the customized enzyme composition dose, if necessary.

According to one embodiment, the at least one database stores and maintains data related to enzyme composition test results. Such test result data may relate to recent technical characterization data. Such test result data may also relate to indirect classifiers of the potential use in specific animal production conditions.

According to one embodiment, the at least one database stores and maintains data related to animal species for which an enzyme composition may be formulated. Such animal species data may relate to physiological needs (including enzyme needs) of an animal species. Such physiological needs may impact nutritional needs that vary depending on species and animal life stage. The animal species data may also include data related to animal species efficiency of use of an enzyme composition. Such data may relate to positive impacts that inclusion of an enzyme composition may have on a specific animal species at a specific growth stage.

According to one embodiment, the at least one database stores and maintains data related to matrization adjustments for animal environmental conditions and energy efficiency, manufacturing and storage conditions of animals and their feed. Such environmental data may impact enzyme choice, usage and interaction.

According to one embodiment, the at least one database stores and maintains data related to enzyme-nutrient interactions specifying safety margins, prices and availability of raw feed materials.

According to one embodiment, the at least one database stores and maintains data related to dosage rules. Such dosage data may related to minimum activation levels and mobile doses, definition of relationships between zootechnical indicators and enzyme functionality. Such dosage data may also relate to minimum activation in terms of substrate concentration, a maximum of functionality by substrate saturation and the levels of enzyme composition that a feed supplier uses for overdosing.

According to one embodiment, the data as provided herein is entered into or received into a cloud pipeline system. According to one embodiment, the cloud pipeline system includes at least one server, processor and memory to receive, analyze, process, detect anomalies and store the various data as provided herein. According to one embodiment, the cloud pipeline system includes more than one or a plurality (e.g., cluster) of servers receive, process and store the various data as provided herein. According to one embodiment, the cloud pipeline system includes at least one computer to receive, analyze, process, detect anomalies, and store the various data as provided herein. According to one embodiment, at least one server are configured to receive, process and store the various data as provided herein. The number of servers, computers or a combination thereof is scalable and varies depending on data size.

According to one embodiment, one or more statistical or machine learning models may be utilized to process the data as provided herein. According to one embodiment, the statistical model is one more of lineal regression, logistical regression, logarithmical regression, survival analysis, anovas, principal competent analysis, ARIMA, or any combination thereof. According to one embodiment, the machine learning model is one or more of decision trees, regression trees, random forest, gradient boosting machine, support vector machine, neural network bayesian network, or any combination thereof.

The methods provided herein may utilize an integrated software system that is adapted to generate altered or otherwise customized enzyme composition recommendations through analysis of one or more variables and data sources described herein. The integrated software system may utilize one or more algorithms that follow a logical structure. The logical structure may take into consideration a variety of factors or data sources described herein.

According to one embodiment, the system includes an animal owner interface. According to one embodiment, the system includes an animal feed producer interface. The aforementioned interfaces may include a data entry system. The animal owner interface may be in wireless communication with at least one server. According to one embodiment, the animal owner interface is in wireless communication with animal feed or enzyme composition manufacturing equipment. According to one embodiment, a feed manufacturing apparatus is in wireless communication with at least one server, processor and memory. According to one embodiment, an enzyme composition manufacturing apparatus is in wireless communication with at least one server, processor and memory.

### Methods

The methods provided herein are suited to aid in identifying enzymes and evaluating enzymes suitable for animal consumption. The methods provided herein are particularly suited in aiding in achieving one or more zootehcnical objectives. Such an objective may include improving the health and performance of an animal.

The methods provided herein may also be utilized to identify enzyme doses. The methods provided herein may evaluate a plurality of variables including the potential nutritional variation generated by the enzymes and the interactions with storage and production conditions exhibited to produce a customized enzyme composition. Other variables considered include, but are not limited to, the characterization of any supporting feed substrate, the coherent definition of minimum of activation, and the expected maximum of functionality. Such variable information may be integrated into a diet formulation software, which will be fed from a data system that, being supported by evidence, constant laboratory analysis, digital applications, production monitoring and real-time feedbacks, enables the mitigation of potential risks associated with nutritional decisions.

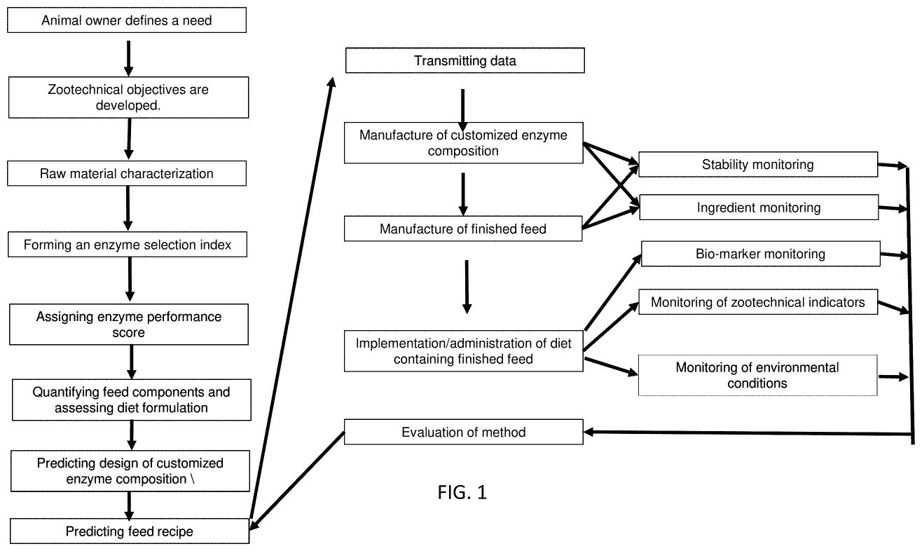

A method of formulating a custom enzyme composition is provided. One embodiment of a method of formulating a customized animal feed is illustrated in FIG. 1.

According to one embodiment, the method of formulating a customized animal feed includes the step of receiving animal owner data. The animal owner data may include objective data related to nutritional needs, animal weight, animal activity level, ammonia production level of the animal, animal body temperature, environmental temperature, humidity, animal weight, animal water intake, animal body pH, or a combination thereof. The animal owner data may be transmitted wirelessly from an animal owners interface to a remote system component such as a database, server, processor or combination thereof.

According to one embodiment, the method of formulating a customized animal feed includes the step of developing one or more health and performance goals based on animal owner needs. According to one embodiment, the method of formulating a customized animal feed includes the step of developing one or more zootechnical objectives. The one or more zootechnical objectives may include any objective obtained via introduction or administration of an enzyme composition that, in turn, may be optionally combined with an animal feed.

According to one embodiment, the method of formulating a customized animal feed includes the step of characterizing raw materials. Raw materials includes enzymes and feed components such as those found in commercially available animal feed.

According to one embodiment, the step of characterizing raw materials aids in projecting or predicting the levels of enzyme hydrolysis that are technically feasible thereby predicting the release of nutrients that can occur at the metabolic level or the potential control that can be exerted on anti-nutritional factors of interest. Raw material characterization may be accomplished by a variety of analyses. According to one embodiment, the raw material characterization may be carried out by near-infrared spectroscopy (NIRS) which includes basic tests such as proximate analysis. The NIRS proximate analysis may aid to define the percentages of moisture, fat, fiber, ash, carbohydrates and protein in a feed component. The NIRS proximate analysis may aid in the quantification of enzyme substrates such as starch (amylases), phytic acid (Phytases) and aid in the determination of the quality of legumes from analysis of favorable components such as amino acids or control components such as trypsin inhibitors. The step of characterizing raw materials allows for the individualization of the general enzyme substrates and aid in quality control of raw materials to verify that such raw materials comply with established specifications or requirements.

According to one embodiment, the step of characterizing raw materials may include performing laboratory analysis via any methodology that facilitates the physical, chemical and microbial characterization of the raw materials. According to one embodiment, the step of characterizing raw materials may include performing simulations of the gastrointestinal tract of an animal to project or predict the functionality of any feed components or additives. Such simulation may predict the impact of feed components or additives on organisms based on the homologation of the metabolic conditions in terms of temperature, pH and presence of endogenous enzymes.

According to one embodiment, the step of characterizing raw materials may include performing in vivo analyses to quantify the response of an animal to being fed raw materials in real conditions, including performance tests, measurement of productive indicators at slaughter, mineralization tests, tissue sampling, body condition score, presence of pathologies, and blood tests/analysis.

According to a particular embodiment, the method includes the steps of analyzing feed production needs, characterizing substrates present in raw materials of the feed, and analyzing commercial enzymes.

The method of formulating a custom enzyme composition may include the step of forming or generating an enzyme selection index. An enzyme selection index may be constructed independently for each enzyme type or category such as for phytases, carbohydrases and proteases. An enzyme selection index ensures repeatability of the system and practicality.

To form an enzyme selection index, suitable enzymes for inclusion in an enzyme composition may undergo various analyses. According to one embodiment, to form an enzyme selection index, suitable enzymes for inclusion in an enzyme composition may undergo stability tests, defined as post-pelletization recoveries, enzyme resistance tests under storage conditions and quantification of activity losses associated with adverse reactions with other raw materials included in a feed formulation or enzyme composition. This analysis will allow for projecting the active principle losses that the customized enzyme composition would have in the productive processes prior to animal consumption.

According to one embodiment, to form an enzyme selection index, suitable enzymes for inclusion in an enzyme composition may undergo physical characterization for the determination of granulometry, particle size uniformity, coating quality, each of which impact feasible to understand factors such as powderiness, statics and ease of distribution in the mixture.

According to one embodiment, to form an enzyme selection index, suitable enzymes for inclusion in an enzyme composition may undergo measurement of activity in different pH conditions that allow projecting enzyme activation in different portions of the gastrointestinal tract. According to one embodiment, to form an enzyme selection index, suitable enzymes for inclusion in an enzyme composition may undergo resistance to adverse metabolic conditions such as the presence of pepsin for quantification of post-consumption losses in animals. According to one embodiment, to form an enzyme selection index, suitable enzymes for inclusion in an enzyme composition may undergo a determination of variables associated with enzyme kinetics for the measurement of affinity for specific substrates and enzyme turnover capacity.

According to one embodiment, to form an enzyme selection index, suitable enzymes for inclusion in an enzyme composition may undergo in vitro and in vivo tests with the resulting data converted into the selection index that allows the identification of the productive conditions in which the enzyme's use would represent a competitive advantage. According to one embodiment, to form an enzyme selection index, suitable enzymes for inclusion in an enzyme composition may undergo in vitro simulations in for the quantification of the hydrolysis potential of substrates or anti-nutritional factors through the summation of reaction products.

According to one embodiment, to form an enzyme selection index, suitable enzymes for inclusion in an enzyme composition may undergo in vivo tests of biomineralization, quantification of metabolites in blood, apparent digestibility, ileal digestibility, among others, to identify the enzyme functionality from the measurement of their direct effects on the animals.

The step of formulating a custom enzyme composition utilizes one or more system components such as a server, processor, database, or a combination thereof to develop the an enzyme selection index. According to one embodiment, one or more statistical or machine learning models may be utilized to process the data.

The method of formulating a custom enzyme composition may include the step of creating and assigning a performance score to an enzyme that qualifies the performance of the enzyme. The step of creating and assigning a performance score to an enzyme may be carried out by one or more system components such as a server, processor, database, or a combination thereof. The score may be calculated by a system component utilizing one or more algorithms or machine models. The score may be calculated by a system component by assigning different values of importance to each of the analyses or tests described herein. The performance score may aid in collating feed products (e.g., commercial feed) and developing customized enzyme compositions. The steps of generating an enzyme selection index and assigning an enzyme performance score are not capable of being performed by the human brain.

According to one embodiment, the weight of the total score can be modified as needed in order to qualify one or more enzyme compositions with respect to the specific requirements presented by an animal owner based on enzyme production conditions. According to one embodiment, feed production needs may be based on animal feed producer input (e.g., feed characteristics needs). The animal producer input may be fed to software and databases provided herein. One or more processors may then be utilized to create, calculate or otherwise assign the performance score.

The method of formulating a custom enzyme composition may include the step of quantifying feed components evaluating enzyme characteristics of enzymes that may be utilized in enzyme compositions, and assessing diet formulation for maximum efficiency. The steps of quantifying feed components evaluating enzyme characteristics of enzymes that may be utilized in enzyme compositions, and assessing diet formulation for maximum efficiency may be carried out by one or more of the system components provided herein include, but not limited to, one or more processors. The step of assessing diet formulation for maximum efficiency may consider any data obtained from the step of characterizing the substrates in the raw materials of the feed and considering a production needs based on a target animal. The step of quantifying feed components and evaluating enzyme characteristics of enzymes that may be utilized in enzyme compositions may consider data related to commercial enzymes available and from the characterization of substrates present in raw materials of the feed. Any data from the commercial enzymes available and characterization of substrates present in raw materials of the feed may be fed into software, processors and databases along with data obtained from quantifying feed components and assessing diet formulation for maximum efficiency.

The method of formulating a custom enzyme composition includes the step of predicting a custom enzyme composition that may be beneficial to the animal. The step of predicting a custom enzyme composition includes the step of processing all data related to the zootehcnical objectives, raw material characterization, enzyme performance score, and feed components/diet formulation. The step of predicting a custom enzyme composition utilizes one or more system components such as a server, processor, database, or a combination thereof to develop the prediction for the custom enzyme composition that may be beneficial to the animal. According to one embodiment, one or more statistical or machine learning models may be utilized to process the data related to the zootehcnical objectives, raw material characterization, enzyme performance score, and feed components/diet formulation. The prediction steps may cannot be performed by a human brain.

The method of formulating a custom enzyme composition includes the step of predicting an animal feed recipe that may be beneficial to the animal. The step of predicting animal feed recipe includes the step of processing all data related to the zootehcnical objectives, raw material characterization, enzyme performance score, feed components/diet formulation and enzyme performance score. The step of predicting animal feed recipe utilizes one or more system components such as a server, processor, database, or a combination thereof to develop the prediction for the customized animal feed recipe that may be beneficial to the animal. According to one embodiment, one or more statistical or machine learning models may be utilized to process the data related to the zootehcnical objectives, raw material characterization, enzyme performance score, and feed components/diet formulation. The prediction steps cannot be performed by a human brain.

According to one embodiment, the method of formulating a custom enzyme composition includes the step of transmitting data related to the customized animal feed recipe. The data may be transmitted wirelessly from a user interface directly to an animal feed manufacturer. The customized animal feed manufacturer may, in turn, receive the customized animal feed recipe directly on an animal feed manufacturer user interface.

According to one embodiment, the method of formulating a custom enzyme composition may include the step of transmitting data related to the customized animal feed recipe to an animal owner for approval or rejection prior to transmission to the customized animal feed manufacturing facility. According to one embodiment, the animal owner may accept or approve the customized animal feed recipe. Upon acceptance, the customized animal feed recipe may be transmitted to the customized animal feed manufacturer. According to one embodiment, the animal owner may reject the customized animal feed recipe. At rejection, the animal owner may provide input regarding modifications or revisions to the customized animal feed recipe. Upon rejection, a signal may be transmitted to back to a remote location housing system components to undertake one or more steps set forth in FIG. 1 to generate a new ore revised feed recipe. All transmissions between the animal owner and any remote location, including the customized animal feed manufacturer, may be conducted wirelessly and sent or received by user interfaces.

According to one embodiment, the method of formulating a custom enzyme composition further includes the step of producing or manufacturing a customized enzyme composition. To manufacture the customized enzyme composition, one or more enzymes must be extracted or otherwise produced. The customized enzyme composition may be formulated as an additive or pre-mix that is added to a finished feed from a feed production facility. The customized enzyme composition may be optionally added to a commercial feed or customized feed.

According to one embodiment, the method of formulating a custom enzyme composition further includes the step of producing or manufacturing the customized animal feed that includes one or more feed components and customized enzyme composition.

A variety of factors may be analyzed and evaluated when manufacturing the customized enzyme composition and animal feed. Such factors include stability of the one or more enzymes as well as stability and type of ingredients in the feed. Such factors may be monitored in real-time with one or more sensors. By monitoring such factors, enzyme functionality and nutrient requirements of the animals may be continuously evaluated. A step of analyzing enzyme recovery may be conducted based on any feed produced at the manufacturing facility. Data produced from the analysis of the enzyme recovery along with any other accompanying data related to the feed manufacturing may be fed back to the software and databases described herein. A step of analyzing temperature impact on feed manufacturing may also be conducted utilizing temperature sensors at the feed manufacturing facility. Any data related to temperature impact on feed manufacturing may be fed back to the software and databases described herein.

According to one embodiment, the method of formulating a custom enzyme composition includes the step of administering the customized animal feed to an animal. The animal may be in need of improvement in health and performance. The step of administration may occur via directly feeding the animal via an appropriate route acceptable for the specific animal species. For example, for livestock, the customized animal feed may be administered by introducing the feed to a dedicated feed trough.

According to one embodiment, the customized enzyme composition administration dose in the customized animal feed may be flexible/variable and not fixed, but will be defined through the quantification of the potential substrate of the raw materials and the interactions that the enzyme and raw materials may have with other nutrients or physiological conditions of the animals that will consume the feed containing the customized enzyme composition. According to one embodiment, a nutritional assessment of the customized enzyme composition and accompanying feed may depend on the nutrient release or nutrient saving potential resulting from the interaction between enzyme compositions and specific raw materials in the feed and not only on data obtained from real-time observation of an owner's animals.

According to one embodiment, the method of formulating a customized animal feed includes the step of evaluating the animal health and performance after administration of the customized enzyme composition. A variety of factors may be analyzed and evaluated during evaluation of the impact on the one or more animals as illustrated in FIG. 1. Such factors include data from in vitro enzyme behavior, interactions in feed manufacturing conditions, storage, and functionality in animal metabolism, bio-marker monitoring, zootechnical indicator monitoring and environmental conditions. Such factors may be monitored in real-time or close to real-time. One factor includes animal weight that may be monitored in real-time or close to real time via scales with sensors that transmit weight data to a central, customized software system for further processing. Animal activity level, ammonia production level of the animal, animal body temperature, environmental temperature, humidity, animal weight, animal water intake, or animal body pH may also be monitored after administration. The data related to the aforementioned factors may be evaluated to improve the diagnosis and design of the customized enzyme composition and animal feed. Specifically, all data derived from the administration of the customized enzyme compositions (including animal owner feedback) may be integrated into a diet formulation platform that simultaneously uses the concept of formulation at minimum cost and maximum functionality to define an enzyme composition. Based on the data derived from the use of the customized enzyme, the concepts of subadditivity between enzyme profiles, minimum of activation and maximum of functionality of each commercial product can be specified for a rigorous determination of the inclusion doses and the expected nutritional contributions of the customized enzyme composition.

According to one embodiment, the method of formulating a customized animal feed includes the step of transmitting evaluation data back to one or more system components for further processing. According to one embodiment, any data related to evaluation of the method as applied to an animal may be fed or otherwise transmitted to one or more system components (e.g., processor) to process and predict beneficial modifications to the customized enzyme composition and animal feed. According to one embodiment, any data related to evaluation of the method as applied to an animal may be fed or otherwise transmitted from an animal owner interface (by an animal owner) to a remote server so that the data may be processed to predict modifications to the customized enzyme composition and animal feed. The animal owner data may relate to animal activity level, ammonia production level of the animal, animal body temperature, environmental temperature, humidity, animal weight, animal water intake, or animal body pH.

According to one embodiment, any data related to evaluation of the method as applied to an animal may be fed or otherwise transmitted to a pre-determined veterinarian to recommend additional interventions such as potential therapeutic components added to a diet/nutritional, genetic, a therapeutic treatment regimen, or a combination thereof. Upon confirmation of acceptance of the additional interventions, a wireless signal is sent to a third party such as, for example, the customized enzyme composition producer. Upon acceptance, instructions may be sent to the third party to manufacture and, optionally, ship the customized enzyme composition to the animal owner. According to one embodiment, the manufacturer may adjust various nutritional components in feed such as, for example, enzyme type, vitamin content, mineral content, caloric content, and protein content.

According to one embodiment, any loss of enzyme activity, under storage conditions and critical thermal treatments such as pelleting, may be quantified for the adjusted determination of enzyme and feed doses in the final feed recipe. Thermal processes are usually present in the production of animal feed as such thermal processes enable microbial control, reduction of feed losses and increase of digestibility of some nutritional components. Despite the advantages, the use of high temperatures in the manufacturing processes are an important factor to consider when evaluating the stability of the feed additives included in a customized enzyme composition. Other manufacturing variables such as the time that the feed remains in the conditioning equipment and the level of friction that the feed presents when passing through the die of the pelletizer or extruder are also factors that may be considered. Therefore, sensors directly adapted to the pelletizers may transmit or otherwise communicate internal temperatures throughout the process, ensuring that the enzymes are not exceeding their thermal tolerance limits and, in case this happens, the enzyme dosage may be adjusted to ensure that the losses are properly covered.

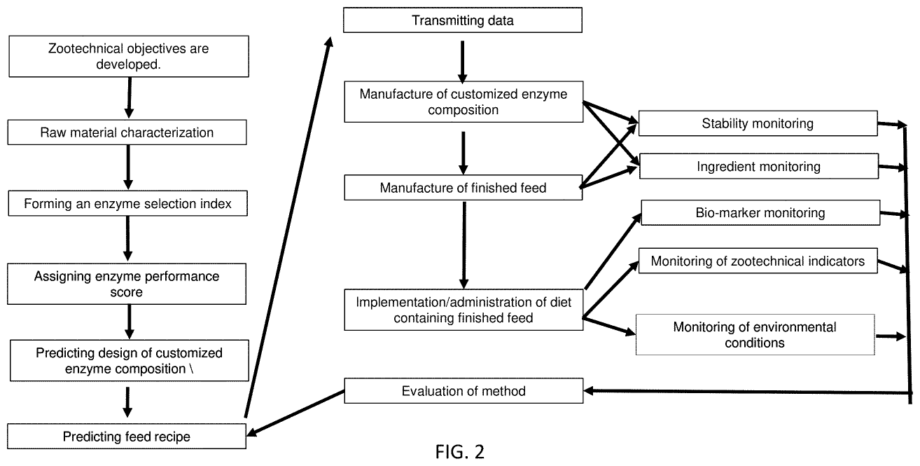

According to one embodiment, any potential enzymes of potential use as provided herein may be evaluated via in vitro and/or in vivo conditions through tests that enable the characterization of functionality conditionals including, but not limited to, quantification of thermostability, enzyme kinetics, resistance to pH conditions and animal performance. According to one embodiment, a method of achieving one or more zootechnical objectives for an animal is illustrated in FIG. 2. According to one embodiment, the method of achieving one or more zootechnical objectives for an animal includes the step of developing one or more zootechnical objectives to be obtained based on administration of an animal feed comprising one or more feed components and a customized enzyme composition. According to one embodiment, the method of achieving one or more zootechnical objectives for an animal includes the step of analyzing and selecting, by processor, one or more feed components and enzymes for suitability in achieving the one or more zootechnical objectives (including analyzing and selecting feed components and enzymes based on nutritional intake). According to one embodiment, the method of achieving one or more zootechnical objectives for an animal includes the step of comparing enzyme characteristics of commercially available enzymes for inclusion in customized enzyme composition. According to one embodiment, the method of achieving one or more zootechnical objectives for an animal includes the step of analyzing and selecting, by processor or other suitable system component, one or more feed components and enzymes for suitability in achieving the one or more zootechnical objectives (including analyzing and selecting feed components and enzymes based on nutritional intake). According to one embodiment, the method of achieving one or more zootechnical objectives for an animal includes the step of predicting, by processor or other suitable component, the customized enzyme composition including one or more enzymes for suitability in achieving the one or more zootechnical objectives. According to one embodiment, the method of achieving one or more zootechnical objectives for an animal includes predicting, by processor or other suitable system component, a recipe for an animal feed including one or more feed components and the customized enzyme composition. According to one embodiment, the method of achieving one or more zootechnical objectives for an animal includes the step of manufacturing the customized enzyme composition. According to one embodiment, the method of achieving one or more zootechnical objectives for an animal includes the step of manufacturing the customized animal feed. According to one embodiment, the method of achieving one or more zootechnical objectives for an animal includes the step of feeding the customized animal feed to at least one animal. According to one embodiment, the method of achieving one or more zootechnical objectives for an animal includes the step of evaluating the impact on the one or more zootechnical objectives based on feeding the customized animal feed to the at least one animal. According to one embodiment, the method of achieving one or more zootechnical objectives for an animal includes the method optionally includes the step of implementing one or more design changes to the customized enzyme composition, feed components, or a combination thereof based on the step of evaluating impact on the one or more zootechnical objectives. According to one embodiment, the method of achieving one or more zootechnical objectives further includes the step of transmitting the customized animal feed recipe to the animal owner for acceptance or denial by the animal owner, wherein upon approval by the animal owner, the customized animal feed recipe is transmitted to an animal feed manufacturer.

A variety of factors may be analyzed and evaluated during evaluation of the impact on the one or more animals as illustrated in FIG. 2. Such factors include data from in vitro enzyme behavior, interactions in feed manufacturing conditions, storage, and functionality in animal metabolism, bio-marker monitoring, zootechnical indicator monitoring and environmental conditions. Such factors may be monitored in real-time or close to real-time. One factor includes animal weight that may be monitored in real-time or close to real time via scales with sensors that transmit weight data to a central, customized software system for further processing. Animal activity level, ammonia production level of the animal, animal body temperature, environmental temperature, humidity, animal weight, animal water intake, or animal body pH may also be monitored after administration. The data related to the aforementioned factors may be evaluated to improve the diagnosis and design of the customized enzyme composition and animal feed.

A method for improving health, performance, or a combination thereof in an animal is provided. The method for improving health, performance, or a combination thereof in an animal may include the step of administering a customized enzyme composition to an animal in need of treatment. The customized enzyme composition may be designed and manufactured according to one or more of the steps set forth in FIG. 1. The method for improving health, performance, or a combination thereof in an animal may include the step of admixing a customized enzyme composition with animal feed that, in turn, optionally incudes one or more feed additives. The method for improving health, performance, or a combination thereof in an animal may include the step of evaluating health, performance, or a combination thereof after administration. Any data related to the evaluated health, performance, or a combination thereof may be fed back or otherwise transmitted to one or more system components (e.g., processor) to process and predict modifications to the customized enzyme composition that may be beneficial to improving health, performance, or a combination thereof.

A method of promoting enzyme utilization in an animal is provided. The method of promoting enzyme utilization in an animal may include the step of administering a customized enzyme composition to an animal in need of treatment. The customized enzyme composition may be designed and manufactured according to one or more of the steps set forth in FIG. 1. The method of promoting enzyme utilization in an animal may include the step of admixing a customized enzyme composition with animal feed that, in turn, optionally incudes one or more feed additives. The method of promoting enzyme utilization in an animal may include the step of evaluating enzyme utilization in the animal. Any data related to enzyme utilization may be fed or otherwise transmitted to one or more system components (e.g., processor) to process and predict modifications to the customized enzyme composition that may be beneficial to improving enzyme utilization.

A method of manufacturing an enzyme composition for achieving one or more zootechnical objectives is provided. The method includes the step of developing one or more zootechnical objectives to be obtained based on administration of a customized enzyme composition (or corresponding feed including the customized enzyme composition). The method further includes the step of designing an enzyme composition that includes one or more enzymes for suitability in achieving the one or more zootechnical objectives. The method further includes the step of predicting or generating a recipe for an enzyme composition. The method may further include the step of manufacturing the customized enzyme composition for achieving one or more zootechnical objectives. The customized enzyme composition may be designed and manufactured according to one or more of the steps set forth in FIG. 1. According to one embodiment, the one or more zootechnical objectives includes improving animal health and performance.

The methods provided herein may also produce a variety of deliverable products or outputs. Such an output includes a bill of materials that may be provided to an animal owner. Such a bill of materials may include one or more of the following: a complete formula for a finished animal feed or customized enzyme composition intended for the feeding of a particular animal species at a specific physiological stage; detail listing of the macro-ingredients; detail listing of the micro-ingredients; detail listing of the one or more enzymes. According to another embodiment, the output includes an economic analysis in which the cost of manufacturing the feed and the expected return on investment for that diet, under the scenario of using enzymes, is projected. According to another embodiment, the output includes a graphical visualization of the potential enzyme substrates and anti-nutritional factors of interest that the feed and enzyme composition would have as well as any expected changes with the use of the recommended feed and enzyme composition. According to one embodiment, any output would be capable of being downloaded in different formats depending on the animal owner's request.

### Enzyme Compositions

According to one embodiment, the customized enzyme compositions provided herein may be combined or otherwise mixed with a compatible animal feed and delivered to an animal in need. According to one embodiment, the customized enzyme compositions provided herein may be combined or otherwise mixed with a compatible animal feed and, optionally, at least one carrier, additive or a combination thereof.

According to one embodiment, the customized enzyme compositions may be introduced or otherwise admixed with a commercially produced feed. The customized enzyme compositions as provided herein increase the bioavailability of nutrients in existing feed (e.g., commercially available feed). The customized enzyme compositions as provided herein may also improve consistency and nutritional value of the feed, improve food efficiency, increase digestibility, improve animal health and performance, and reduce the effect of anti-nutrients in an animal's diet. The customized enzyme compositions, once fed to an animal, may improve an animal's energy level and overall muscle mass. For animals, such as livestock, the customized enzyme compositions may result in increased yield and profitability by reducing the cost of feed the animals.

The enzymes included in the customized enzyme compositions provided herein include, but are not limited to, carbohydrases, proteases and phytases. The enzymes included in the customized enzyme compositions provided herein may be obtained from animals, plants or microorganisms.

The carbohydrases act on various substrates in animal feed such as fiber and starch. The carbohyrases improve digestibility of plant biomass and increase energy. Suitable carbohydrases include, but are not limited to, xylanases, β-glucanases, β-mannanases, pectinases, α-galactosidases, and α-amylase. The proteases increase nitrogen retention and improve protein digestibility as well as hydrolysis of dietary proteins. Suitable poteases include, but are not limited to, chymosin, pepsin A bromelain, papain, ficine, aminopeptidase, bacillolysin 1, dipeptidyl peptidase III, chymotrypsin, subtilisin, and trypsin. The phytases degrade phytate bonds releasing trapped nutrients and increases the absorption of phosphorus and amino acid availability. The phytases include, but are not limited to, acid phytases of histidine.

Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be obvious to those skilled in the art, and all modifications that do not depart from the spirit of the disclosure are intended to be included with the scope of the appended claims.

### PROPHETIC EXAMPLE

An experimental field trial may be conducted to evaluate the predictive capacity of the methods provided herein and the effect of enzyme compositions on performance, energy efficiency and biomarker production in broilers. To this end, ten different treatments may be formulated in which the same population of animals and fed feeds containing different raw materials, different amounts of minerals and heterogeneous enzyme packages, with weekly measurements of performance indicators, energy use efficiency and laboratory sampling. The experiment would be expected to show: (i) it may be possible to select an adjusted and higher value enzyme composition regimen if the potential substrates available in the diet are known; (ii) it may be possible to determine differences in energy efficiency of birds fed different enzyme compositions; and (iii) which biomarker is most related to enzyme functionality under on-farm production conditions.

### Experimental Example 1

### Pig Fattening

An experimental field trial was carried out to evaluate the predictive ability of the methods provided herein and the effect of customized enzyme compositions on animal health and performance. This experimental field trial was also conducted to compare a traditional enzymatic composition administration (i.e., traditional enzyme packages commercially available in the market) with the customized enzyme compositions predicted and defined by the system and methods provided herein. The predicted customized enzyme composition was based, in part, on the analysis of substrate potential in normal conditions, in growing-finishing pigs.

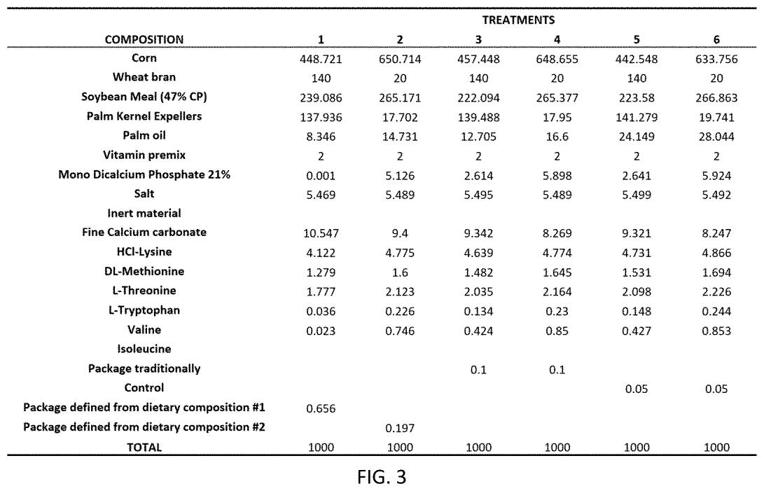

For this purpose, six treatments were formulated in which the same population of animals was fed with animal feed containing the same nutritional restrictions but with differences in the inclusions of raw materials and with heterogeneous customized enzyme compositions. The six treatments are set forth in Table 1. Weekly measurements of health and performance indicators from the weighing of the animals was gathered. A description on each treatment diet is illustrated in FIG. 3 (note: “package” refers to the customized enzyme composition for treatment 1 and treatment 2 in FIG. 3).

TABLE 1
Treatments

| | Treat- | Treat- | Treat- | Treat- | Treat- | Treat- |
|---|---|---|---|---|---|---|
| | ment | ment | ment | ment | ment | ment |
| Item | 1 | 2 | 3 | 4 | 5 | 6 |
| Fiber | 20% | 10% | 20% | 10% | 20% | 10% |
| | High | Low | High | Low | High | High |
| Enzyme | Cus- | Cus- | Traditional | Traditional | Control | Control |
| | tomized | tomized | enzyme | enzyme | (Phytase | (Phytase |
| | Enzyme | Enzyme | package | package | only) | only) |
| | Com- | Com- | | | | |
| | position | position | | | | |

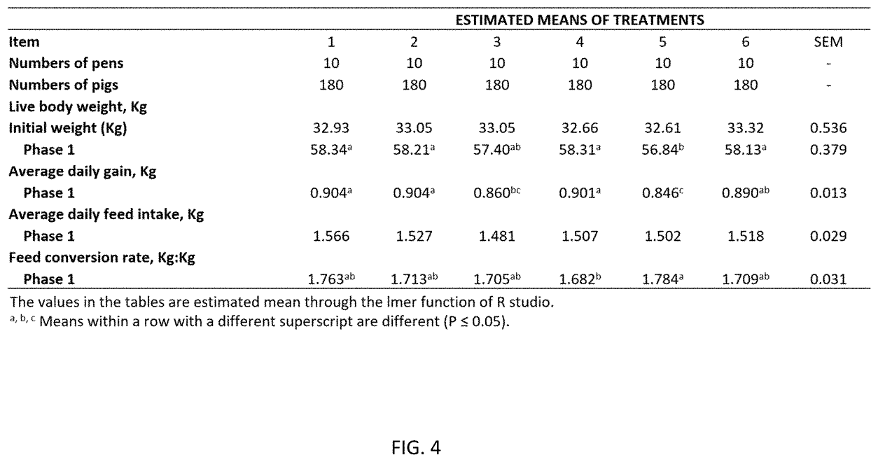

The present example illustrated the system and methods' ability to select an adjusted and higher value customized enzyme composition regime if the potential substrates available in the diet are known. The present example also illustrated the system and methods' ability to formulate lower cost diets by including more fibrous raw materials when using enzyme compositions that allow more fibrous raw material utilization. The present example also illustrated that indicators associated with pig production can be improved if customized enzyme compositions are formulated consistent with the raw materials used. Further detail regarding the results on various physical indicators for the pigs is illustrated in FIG. 4.

### Experimental Example 2

### Enzyme Selection Index Capacity—Broilers

An experimental field trial was carried out to evaluate the comparative capacity of the enzyme selection index, in order to identify phytase-type commercial products with homologous activity and with greater competitiveness in terms of costs. Three treatments (Diet 1, Diet 2 and Diet 3) were formulated in which the same population of animals were fed (broilers) with the same animal feed in which only the commercial brand of the phytase used was modified and the impact of these diets on the microbiome and intestinal health was evaluated. The three treatments/diets are set forth in Table 2.

TABLE 2
Treatments/Diets

| Ingredients (%) | Diet 1 | Diet 2 | Diet 3 |
|---|---|---|---|
| Basal Premix | 1.513 | 1.513 | 1.513 |
| Phytase 1 | 0.0183 | 0 | 0 |
| Phytase 2 | 0 | 0.0183 | 0 |
| Phytase 3 | 0 | 0 | 0.0183 |

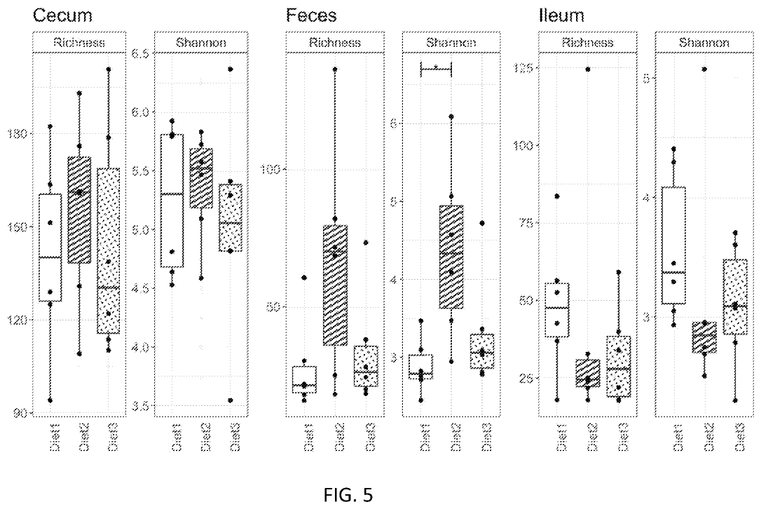

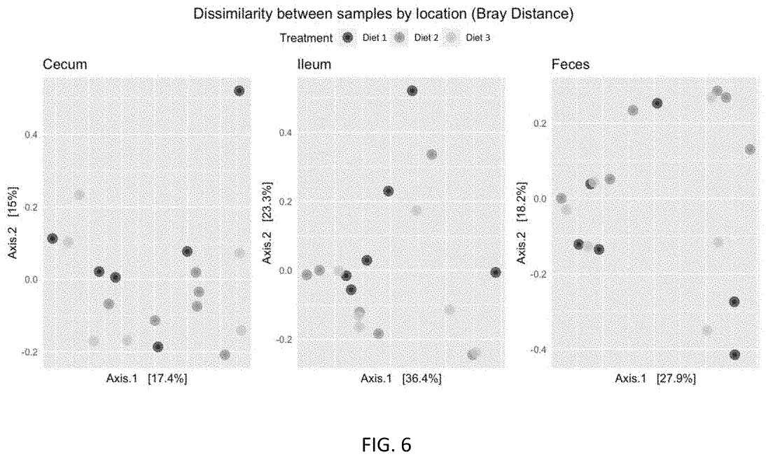

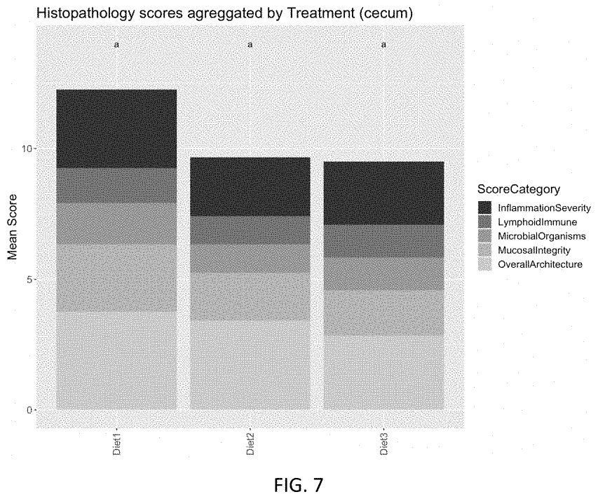

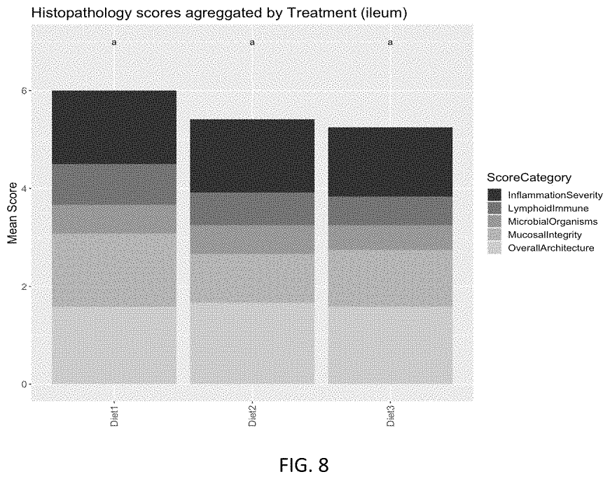

The present example illustrated how characterizations of the enzymes to standardize their effect on the microbiome and intestinal health is possible. The present example illustrated how the system and methods may be utilized to use more competitive cost phytases without affecting results in the animals. Specifically, the least expensive (Diet 2) was shown to increase alpha diversity in feces compared to Diet 1 as illustrated in FIG. 5. The general composition of the intestinal microbiome in terms of beta diversity did not change between diets with different phytases, which may indicate that the change of commercial brand is not generating an imbalance in the intestinal ecosystem (see FIG. 6). In histopathology, the two least expensive phytases improves intestinal tissue quality as illustrated in the histopathology scores (aggregated) in FIG. 7 (cecum) and FIG. 8 (ileum).

### Generalized Statements of the Disclosure

The following numbered statements provide a general description of the disclosure and are not intended to limit the appended claims.

Statement 1. The present disclosure provides a method of achieving one or more zootechnical objectives, the method comprising:

developing one or more zootechnical objectives to be obtained based on administration of an animal feed comprising one or more feed components and a customized enzyme composition;

analyzing and selecting, by processor, one or more feed components and enzymes for suitability in achieving the one or more zootechnical objectives, including nutritional intake;

predicting, by processor, the customized enzyme composition comprising one or more enzymes for suitability in achieving the one or more zootechnical objectives;

predicting, by processor, a recipe for an animal feed comprising one or more feed components and the customized enzyme composition;

manufacturing the customized enzyme composition;

manufacturing the customized animal feed;

feeding the customized animal feed to at least one animal;

evaluating impact on the one or more zootechnical objectives based on feeding the customized animal feed to the at least one animal; and

optionally, implementing one or more design changes to the customized enzyme composition, feed components, or a combination thereof based on the step of evaluating impact on the one or more zootechnical objectives.

Statement 2. The present disclosure provides a method of statement 1, wherein the one or more zootechnical objectives includes improving animal performance.

Statement 3. The present disclosure provides a method of statements 1-2, wherein the customized animal feed and enzyme composition are combined prior to feeding to the at least one animal.

Statement 4. The present disclosure provides a method of statements 1-3, wherein the step of evaluating impact on the one or more zootechnical objectives includes the step of analyzing, by processor, animal weight, animal activity level, ammonia production level of the animal, animal body temperature, environmental temperature, humidity, animal weight, animal water intake, animal body pH, or a combination thereof.

Statement 5. The present disclosure provides a method of manufacturing an enzyme composition for achieving one or more zootechnical objectives, the method comprising:

developing one or more zootechnical objectives to be obtained based on administration of an animal feed comprising one or more feed components and a customized enzyme composition;

analyzing and selecting, by processor, one or more feed components and enzymes for suitability in achieving the one or more zootechnical objectives;

predicting, by processor, the customized enzyme composition comprising one or more enzymes for suitability in achieving the one or more zootechnical objectives;

predicting, by processor, a recipe for an animal feed comprising one or more feed components and the customized enzyme composition; and

manufacturing the customized enzyme composition for achieving one or more zootechnical objectives, upon administration.

Statement 6. The present disclosure provides a method of statement 5, wherein the one or more zootechnical objectives includes improving animal health and performance.

Statement 7. The present disclosure provides an animal feed or animal feed additive that includes an enzyme composition of statements 5-6.

Statement 8. The present disclosure provides an enzyme composition for feeding to an animal to achieve one or more zootechnical objectives, the customized enzyme composition comprising

at least one enzyme; and

optionally, one or more suitable carriers,

wherein the at least one enzyme is selected to achieve the one or more zootechnical objectives.

Statement 9. The present disclosure provides a system for achieving one or more zootechnical objectives comprising

at least one server;

at least one database;

at least one software package;

at least one animal sensor coupled to a gateway that is in wireless communication with the at least one server, database and software package;

a memory and processor in wireless communication with the server, database, software package and sensor, the memory and processor configured to perform the steps of statements 1, 5, 10, 12 or 15.

Statement 10. The present disclosure provides a system of statement 9, wherein the software package is adapted to run at least one algorithm to assist in:

designing an enzyme composition comprising one or more enzymes for suitability in achieving the one or more zootechnical objectives;

analyzing and selecting, by processor, one or more feed components and enzymes for suitability in achieving the one or more zootechnical objectives;

predicting, by processor, the customized enzyme composition comprising one or more enzymes for suitability in achieving the one or more zootechnical objectives;

predicting, by processor, a recipe for an animal feed comprising one or more feed components and the customized enzyme composition;

evaluating impact on the one or more zootechnical objectives based on feeding the customized animal feed regimen or recipe to the at least one animal; or

a combination thereof.

Statement 11. The present disclosure provides a system of statements 9-10, further comprising an animal owner's interface including a data entry system.

Statement 12. The present disclosure provides a method of achieving one or more zootechnical objectives, the method comprising:

developing one or more zootechnical objectives to be obtained based on administration of an animal feed, the customized animal feed comprising one or more feed components and a customized enzyme composition;

characterizing feed raw materials, the raw materials including one or more feed components and one or more enzymes;

generating, by processor, an enzyme selection index;

assigning, by processor, an enzyme performance score;

predicting, by processor, the customized enzyme composition comprising one or more enzymes for suitability in achieving the one or more zootechnical objectives;

predicting, by processor, a recipe for the customized animal feed;

manufacturing the customized enzyme composition;

manufacturing the customized animal feed;

administering the customized animal feed to an animal;

evaluating the impact on the one or more zootechnical objectives after administration of the customized animal feed; and

optionally, implementing one or more design changes to the customized enzyme composition, feed components, or a combination thereof based on the step of evaluating impact on the one or more zootechnical objectives.

Statement 13. The present disclosure provides a method of statement 12, wherein the one or more zootechnical objectives includes improving animal performance.

Statement 14. The present disclosure provides a method of statements 12-13, wherein the step of evaluating impact on the one or more zootechnical objectives includes the step of analyzing, by processor, animal weight, animal activity level, ammonia production level of the animal, animal body temperature, environmental temperature, humidity, animal weight, animal water intake, animal body pH, or a combination thereof.

Statement 15. The present disclosure provides a method of formulating a customized animal feed, the method comprising the steps of:

receiving animal owner data;

developing one or more zootechnical objectives to be obtained based on administration of an animal feed, the customized animal feed including one or more feed components and a customized enzyme composition;

characterizing feed raw materials, the raw materials including one or more feed components and one or more enzymes;

generating, by processor, an enzyme selection index;

assigning, by processor, an enzyme performance score;

predicting, by processor, the customized enzyme composition comprising one or more enzymes for suitability in achieving the one or more zootechnical objectives;

predicting, by processor, a recipe for the customized animal feed;

manufacturing the customized enzyme composition;

manufacturing the customized animal feed;

administering the customized animal feed to an animal; and

evaluating the animal health and performance after administration of the customized animal feed.

Statement 16. The present disclosure provides a method of statement 15, wherein the animal owner data includes animal nutritional needs, animal weight, animal activity level, ammonia production level of the animal, animal body temperature, ambient temperature of a facility where the animal is kept, humidity, animal weight, animal water intake, animal body pH, or a combination thereof.

Statement 17. The present disclosure provides a method of statements 15-16, wherein the one or more zootechnical objectives includes improving animal health and performance.

Statement 18. The present disclosure provides a method of statements 15-17, further comprising the steps of quantifying feed components, evaluating enzyme characteristics of enzymes that may be utilized in enzyme compositions, and assessing diet formulation for maximum efficiency.

Statement 19. The present disclosure provides a method of statements 1-18, further comprising the step of transmitting the customized animal feed recipe to the animal owner for acceptance or denial by the animal owner, wherein upon approval by the animal owner, the customized animal feed recipe is transmitted to an animal feed manufacturer.

Statement 20. The present disclosure provides a method of statements 1-19, wherein none of the prediction steps are capable of being performed by the human brain.

Statement 21. The present disclosure provides a method of statements 1-20, wherein the steps of generating an enzyme selection index and assigning an enzyme performance score are not capable of being performed by the human brain.

What is claimed is:

1. A method of formulating a customized animal feed, the method comprising the steps of:
receiving animal owner data;
developing one or more zootechnical objectives to be obtained based on administration of a customized animal feed, the customized animal feed comprising one or more feed components and a customized enzyme composition;
characterizing feed raw materials, the raw materials including one or more feed components and one or more enzymes;
generating, by processor, an enzyme selection index;
assigning, by processor, an enzyme performance score;
predicting, by processor, the customized enzyme composition comprising one or more enzymes for suitability in achieving the one or more zootechnical objectives;
predicting, by processor, a recipe for the animal feed;
manufacturing the customized enzyme composition;
manufacturing the customized animal feed;
administering the customized animal feed to an animal; and
evaluating the animal health and performance after administration of the customized animal feed.

2. The method of claim 1, wherein the animal owner data includes animal nutritional needs, animal weight, animal activity level, ammonia production level of the animal, animal body temperature, ambient temperature of a facility where the animal is kept, humidity, animal weight, animal water intake, animal body pH, or a combination thereof.

3. The method of claim 1, wherein the one or more zootechnical objectives includes improving animal health and performance.

4. The method of claim 1, further comprising the steps of quantifying feed components, evaluating enzyme characteristics of enzymes that may be utilized in enzyme compositions, and assessing diet formulation for maximum efficiency.

5. The method of claim 1, further comprising the step of transmitting the customized animal feed recipe to the animal owner for acceptance or denial by the animal owner, wherein upon approval by the animal owner, the customized animal feed recipe is transmitted to an animal feed manufacturer.

6. A method of achieving one or more zootechnical objectives for at least one animal, the method comprising:
developing one or more zootechnical objectives to be obtained based on administration of an animal feed comprising one or more feed components and a customized enzyme composition;
analyzing and selecting, by processor, one or more feed components and enzymes for suitability in achieving the one or more zootechnical objectives;
predicting, by processor, the customized enzyme composition comprising one or more enzymes for suitability in achieving the one or more zootechnical objectives;
predicting, by processor, a recipe for an animal feed comprising one or more feed components and the customized enzyme composition;
manufacturing the customized enzyme composition;
manufacturing the customized animal feed;
feeding or administering the customized animal feed to the at least one animal;
evaluating impact on the one or more zootechnical objectives based on feeding the customized animal feed to the at least one animal; and
optionally, implementing one or more design changes to the customized enzyme composition, feed components, or a combination thereof based on the step of evaluating impact on the one or more zootechnical objectives.

7. The method of claim 6 wherein the one or more zootechnical objectives includes improving animal health and performance.

8. The method of claim 6, wherein the step of evaluating impact on the one or more zootechnical objectives includes the step of analyzing, by processor, animal weight, animal activity level, ammonia production level of the animal, animal body temperature, environmental temperature, humidity, animal weight, animal water intake, animal body pH, or a combination thereof.

9. A system for achieving one or more zootechnical objectives comprising
at least one server;
at least one database;
at least one software package;
at least one animal sensor coupled to a gateway that is in wireless communication with the at least one server, database and software package; and
a memory and processor in wireless communication with the server, database, software package and sensor, the memory and processor configured to perform the steps of claim 1.

10. The system of claim 9, wherein the software package is adapted to run at least one algorithm to assist in:
analyzing and selecting, by processor, one or more feed components and enzymes for suitability in achieving the one or more zootechnical objectives;
predicting, by processor, the customized enzyme composition comprising one or more enzymes for suitability in achieving the one or more zootechnical objectives;
predicting, by processor, a recipe for an animal feed comprising one or more feed components and the customized enzyme composition;
evaluating impact on the one or more zootechnical objectives based on feeding the customized animal feed to the at least one animal; or
a combination thereof.

11. The system of claim 10, further comprising an animal owner's interface including a data entry system.
